(19) United States
(12) Patent Application Publication
(10) Pub. No.: US 2003/0013898 A1
(43) Pub. Date: Jan. 16, 2003

(54) Pericyte protective agent

(75) Inventors: Hiroshi Yamamoto, Kanazawa-shi (JP); Sho-ichi Yamagishi, New York, NY (US); Hajimu Kurumatani, Kamakura-shi (JP); Koichi Hisano, Tokyo (JP); Kazuo Hirano, Iruma-shi (JP)

Correspondence Address: NIXON & VANDERHYE P.C., 8th Floor, 1100 North Glebe Road, Arlington, VA, 22201, US

(73) Assignee: Toray Industries, Inc.

(21) Appl. No.: 10/163,869

(22) Filed: Jun. 7, 2002

Related U.S. Application Data
(63) Continuation of application No. 09/529,386, filed on May 25, 2000, now abandoned.

(30) Foreign Application Priority Data
Aug. 14, 1998 (JP) 244383/98
Aug. 10, 1999 PCT/JP99/04329

(51) Int. Cl. C07D 307/92; C07D 307/93

(52) U.S. Cl. 549/458

(57) ABSTRACT

The present invention offers a pericyte protective agent having a prostanoic acid derivative as the effective component. The pericyte protective agent to which the present invention relates is effective in the prevention and treatment of diabetic retinopathy, diabetic neuropathy, diabetic nephropathy and the like.

### TECHNICAL FIELD

The present invention relates to a protective agent for pericytes present at the periphery of vascular endothelial cells in microvessels.

### TECHNICAL BACKGROUND

Pericytes are cells present surrounding the vascular endothelial cells in microvessels and they act not only in tension regulation in the microvessels but also in the maintenance of endothelial cell functions such as endothelial cell proliferation suppression and prostacyclin production, and they play an important role in the maintenance of microvascular homeostasis.

If disorders of these pericytes occur, the functions of the endothelial cells constituting the blood vessels are impaired and there is thought to be a link with the onset and progress of various vascular disorders such as diabetic retinopathy, diabetic neuropathy, diabetic nephropathy and other such diabetic microvascular disorders, hypertension, arteriosclerosis, peripheral circulatory disturbance (intermittent claudication and the like), cerebrovascular disorders and ischaemic heart disease.

Prostanoic acid derivatives are known to have various physiological activities and they can be divided into several groups according to the modification of the five-membered ring moiety comprising C-8 to C-12. Among these, the compounds where the carbon atoms at positions 6 and 9 are linked via an oxygen atom are referred to as PGI, and prostaglandin I₂ (PGI₂, prostacyclin) is regarded as typical. This PGI₂ is known to be a substance with a powerful platelet aggregation suppressing action and a peripheral blood vessel dilating action (see Nature Vol.268, p.688, 1976).

Furthermore PGI derivatives having a skeletal structure where the exoenol ether moiety structure, which is the characteristic structure of PGI₂, has been converted to the inter-m-phenylene form have been described in JP-B-2-12226, JP-B-2-57548 and JP-B-1-53672 as compounds where the instability of PGI₂ has been substantially improved. Again, in addition to PGI derivatives with the carbon atoms at positions 6 and 9 via an oxygen, there are known PGI derivatives where this oxygen atom has been replaced with a carbon atom or some other heteroatom. Examples include Ataprost, Iloprost, Clinprost, Ciprostene, Naxaprostene, Taprosten, Cicaprost, Pimilprost, CH-169 and CS570 (see Gendai Iryosha, Sosetsu Purosutaguranjin [An Outline of Prostaglandins] No. 1, p123, 1994, Asu no Shinyaku 15-IV-p185, 1996, and Asu no Shinyaku 15-III-p551, 1996).

However, hitherto, drugs capable of maintaining pericyte function have been entirely unknown, and the fact that prostaglandins have a pericyte protecting action has also been completely unknown.

### OBJECT OF THE INVENTION

The present invention offers a pericyte protective agent which, by displaying a vascular pericyte protecting action, is effective in the prevention and treatment of various vascular disorders such as diabetic retinopathy, diabetic neuropathy, diabetic nephropathy and other such diabetic microvascular disorders, hypertension, arteriosclerosis, peripheral circulatory disturbance (intermittent claudication and the like), cerebrovascular disorders and ischaemic heart disease.

### DISCLOSURE OF THE INVENTION

As a result of painstaking research, the present inventors have discovered that prostanoic acid derivatives exhibit an outstanding pericyte protecting action, and the present invention has been perfected based thereon.

Specifically, the present invention offers a pericyte protective agent in which a prostanoic acid derivative is the effective component.

### BRIEF EXPLANATION OF THE DRAWINGS

FIG. 1 shows the cell protection effect of Beraprost sodium on pericytes.

### OPTIMUM FORM FOR PRACTISING THE INVENTION

As aforementioned, the pericyte protective agent of the present invention has a prostanoic acid derivative as its effective component. Here a “prostanoic acid derivative” may be any type of prostaglandin having the prostanoic acid skeletal structure, or derivative thereof. Among these, preferred prostanoic acid derivatives are the prostaglandin I derivatives and, as such prostaglandin derivatives, there may be used prostaglandin I₁ derivatives, prostaglandin I₂ derivatives and prostaglandin I₃ derivatives or pharmacologically acceptable salts thereof, preferably prostaglandin I₂ derivatives or pharmacologically acceptable salts thereof. Still further preferred is the use of the 4,8-inter-m-phenylene prostaglandin I₂ derivatives represented by the following general formula (I)

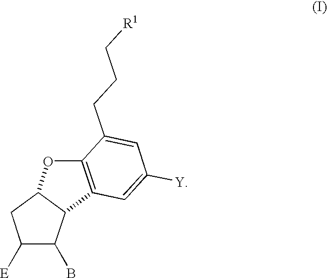

[where R¹ is

(A) COOR²

(where R² is

1) hydrogen or a pharmacologically acceptable cation,

2) a C₁₋₁₂ straight chain alkyl or C₃₋₁₄ branched alkyl,

3) -Z-R³

(where Z is a valence bond or a straight chain or branched alkylene represented by CₜH₂ₜ, where t denotes an integer in the range 1-6, and R³ is a C₃₋₁₂ cycloalkyl or C₃₋₁₂ substituted cycloalkyl which is substituted with from one to three R⁴ groups, and R⁴ denotes hydrogen or a C₁₋₅ alkyl),

4) —(CH₂CH₂O)ₙCH₃

(where n denotes an integer in the range 1 to 5),

5) -Z-Ar¹

(where Z is defined as above and Ar¹ denotes phenyl, α-naphthyl, β-naphthyl, 2-pyridyl, 3-pyridyl, 4-pyridyl, α-furyl, β-furyl, α-thienyl, β-thienyl or substituted phenyl (where the substituent groups are at least one of chlorine, bromine, fluorine, iodine, trifluoromethyl, C₁₋₄ alkyl, nitro, cyano, methoxy, phenyl, phenoxy, p-acetamidobenzamido, —CH═N—NH—C(═O)—NH₂, —NH—C(═O)-Ph, —NH—C(═O)—CH₃ and —NH—C(═O)—NH₂)),

6) —CₜH₂ₜCOOR⁴

(where CₜH₂ₜ and R⁴ are as defined above),

7) —CₜH₂ₜN(R⁴)₂

(where CₜH₂ₜ and R⁴ are as defined above),

8) —CH(R⁵)—C(═O)—R⁶

(where R⁵ denotes hydrogen or benzoyl, and R⁶ denotes phenyl, p-bromophenyl, p-chlorophenyl, p-biphenyl, p-nitrophenyl, p-benzamidophenyl or 2-naphthyl),

9) —CₚH₂ₚ—W—R⁷

(where W is —CH═CH—, —CH═CR⁷— or —C≡C—, and R⁷ is hydrogen or a C₁₋₃₀ straight chain or branched alkyl or aralkyl), and p denotes an integer in the range 1-5), or

10) —CH(CH₂OR⁸)₂

(where R⁸ denotes a C₁₋₃₀ alkyl or acyl),

(B) —CH₂OH

(C) —C(═O)N(R⁹)₂

(where R⁹ represents hydrogen, a C₁₋₁₂ straight chain alkyl, C₃₋₁₂ branched alkyl, C₃₋₁₂ cycloalkyl, C₄₋₁₃ cycloalkylalkylene, phenyl, substituted phenyl (where the substituent groups are as defined in (A) 5) above), C₇₋₁₂ aralkyl or SO₂R¹⁰, and R¹⁰ represents a C₁₋₁₀ alkyl, C₃₋₁₂ cycloalkyl, phenyl, substituted phenyl (where the substituent groups are as defined in (A) 5) above) or a C₇₋₁₂ aralkyl, and the two R⁹ groups may be the same or different, but where one represents —SO₂R¹⁰ then the other R⁹ is not —SO₂R¹⁰), or

(D) —CH₂OTHP (where THP is a tetrahydropyranyl group),

Y is hydrogen, a C₁₋₄ alkyl, chlorine, bromine, fluorine, formyl, methoxy or nitro,

B is —X—C(R¹¹)(R¹²)OR¹³

(where R¹¹ is hydrogen or a C₁₋₄ alkyl, R¹³ is hydrogen, C₁₋₁₄ acyl, C₆₋₁₅ aroyl, tetrahydropyranyl, tetrahydrofuranyl, 1-ethoxyethyl or t-butyl,

X is

1) —CH₂—CH₂—

2) —CH═CH— or

3) —C≡C— and

R¹² represents

1) a C₁₋₁₂ straight chain alkyl or C₃₋₁₄ branched alkyl,

2) -Z-Ar²

(where Z is as defined above and Ar² represents phenyl, α-naphthyl, β-naphthyl or phenyl substituted with at least one of chlorine, bromine, fluorine, iodine, trifluoromethyl, C₁₋₄ alkyl, nitro, cyano, methoxy, phenyl or phenoxy),

3) —CₜH₂ₜOR¹⁴

(where CₜH₂ₜ is as defined above and R¹⁴ represents a C₁₋₆ straight chain alkyl, C₃₋₆ branched alkyl, phenyl, phenyl substituted with at least one of chlorine, bromine, fluorine, iodine, trifluoromethyl, C₁₋₄ alkyl, nitro, cyano, methoxy, phenyl or phenoxy, cyclopentyl, cyclohexyl, or cyclopentyl or cyclohexyl substituted with from one to four C₁₋₄ straight chain alkyls),

4) -Z-R³

(where Z and R³ are as defined above),

5) —CₜH₂ₜ—CH═C(R¹⁵)R¹⁶

(where CₜH₂ₜ is as defined above, and R¹⁵ and R¹⁶ each independently represent hydrogen, methyl, ethyl, propyl or butyl), or

6) —CᵤH₂ᵤ—C≡C—R¹⁷

(where u is an integer in the range 1-7, CᵤH₂ᵤ represents a straight chain or branched alkylene, and R¹⁷ represents a C₁₋₆ straight chain alkyl,

E represents hydrogen or —OR¹⁸ (where R¹⁸ represents a C₁₋₁₂ acyl, C₇₋₁₅ aroyl or R² (where R² is as defined above)),

and where the general formula represents the d-form, l-form or dl-form],

or pharmacologically acceptable salts thereof.

Among the PGI₂ derivatives represented by the aforementioned general formula (I), Beraprost (the general name of (±)-(1R*,2R*,3aS*,8bS*)-2,3,3a,8b-tetrahydro-2-hydroxy-1-[(E)-(3S*)-3-hydroxy-4-methyl-1-octen-6-ynyl]-1H-cyclopenta[b]benzofuran-5-butyric acid), Iloprost, Clinprost, Ataprost, Ciprostene, Naxaprostene, Taprosten, Cicaprost, Pimilprost, CH-169 and SM-10902, and their salts, can be cited as especially preferred but there is no particular restriction thereto.

The prostanoic acid derivatives used in the present invention can be produced by known methods and, for example, the compounds represented by aforementioned general formula (I) can be produced by the method described in JP-B-1-53672.

The pericyte protective agent of the present invention has an outstanding pericyte protecting action and is effective in the prevention and treatment of hypertension and arteriosclerosis where vascular damage is closely involved in the onset and worsening of the disease state, and also of peripheral circulatory disturbance (intermittent claudication and the like), cerebrovascular disorders, ischaemic heart disease and various other such vascular disorders. Furthermore, it is effective in the prevention and treatment of diabetic angiopathy such as diabetic retinopathy, diabetic neuropathy and diabetic nephropathy which are associated with the loss of pericytes due to advanced glycation endproducts (AGE), and also complications of diabetes like arteriosclerosis, ischaemic heart disease, cerebrovascular disorders and other such macrovascular disorders.

The prostanoic acid derivatives of the present invention bring about a marked pericyte protecting effect as a result of oral or parenteral administration.

In the present invention, for adults the prostanoic acid derivatives are administered 1-3 times a day at a dose of 0.1 μg-500 mg/person.

Regarding the pericyte protective agent of the present invention, one or several types of prostanoic acid derivative may be used directly as they are, but oral administration can also be carried out in the form of a solid material containing undermentioned additives.

As examples of additives, there are starches, lactose, sucrose, glucose, mannitol, calcium carbonate, calcium sulphate and other such excipients; starches, dextrin, gum Arabic, tragacanth, methyl cellulose, gelatin, polyvinyl pyrrolidone, polyvinyl alcohol and other such binders; starches, polyvinyl pyrrolidone, crystalline cellulose and other such disintegrants; magnesium stearate, talc and other such lubricants; and colorants and fragrances.

The pericyte protective agent of the present invention can be used in various dosage forms; specifically, as tablets, sugar-coated tablets, powders, granules, troches, capsules, pills, syrups and other such conventionally-used dosage forms.

Furthermore, parenteral administration in the form of a sterilized solution may also be carried out and, moreover, other dissolved substances can also be employed, for example sufficient sodium chloride, glucose or the like, to make the solution isotonic.

The pericyte protective agent of the present invention can be orally or parenterally administered. As parenteral administration routes, there are intravenous injection, subcutaneous injection, intramuscular injection, perintestinal administration, transdermal administration, eye instillation and transnasal administration.

As a specific example of a formulation, there is the following.

| | | |
|---|---|---|
| prostanoic acid derivative | 500 | μg |
| physiological saline | 1 | ml |

### EXAMPLES

Below, the present invention is explained in more specific terms by giving examples, but the present invention is not restricted to the examples provided.

### Reference Example 1

### Preparation of Materials

(1) Preparation of AGE-BSA

AGE-BSA was formed by incubating bovine serum albumin (BSA) (fraction V, fatty acid-free, endotoxin-free, produced by Boehringer Mannheim GmbH, Germany) with 0.5 M glucose at 37° C. for 6 weeks under sterilized conditions. Unbound glucose was removed by dialysing against phosphate-buffered saline (PBS), and the glucose-modified macromolecular substance was purified by heparin-Sepharose CL-4B column (produced by Pharmacia LKB, Uppsala, Sweden) chromatography and used as the AGE-BSA. Control non-glycated BSA was prepared by carrying out incubation under the same conditions as aforementioned, except for not including the glucose. The fact that non-glycated BSA had been separated from the AGE-BSA was confirmed by SDS-PAGE. The AGE-BSA concentration was measured by the Bradford method (Bradford, M.: Anal. Biochem. 72: 248-254, 1976).

### Example 1

### Pericyte Protecting Action

Pericytes were isolated from bovine retina and maintained in Dulbecco's modified Eagle medium containing 20% foetal calf serum.

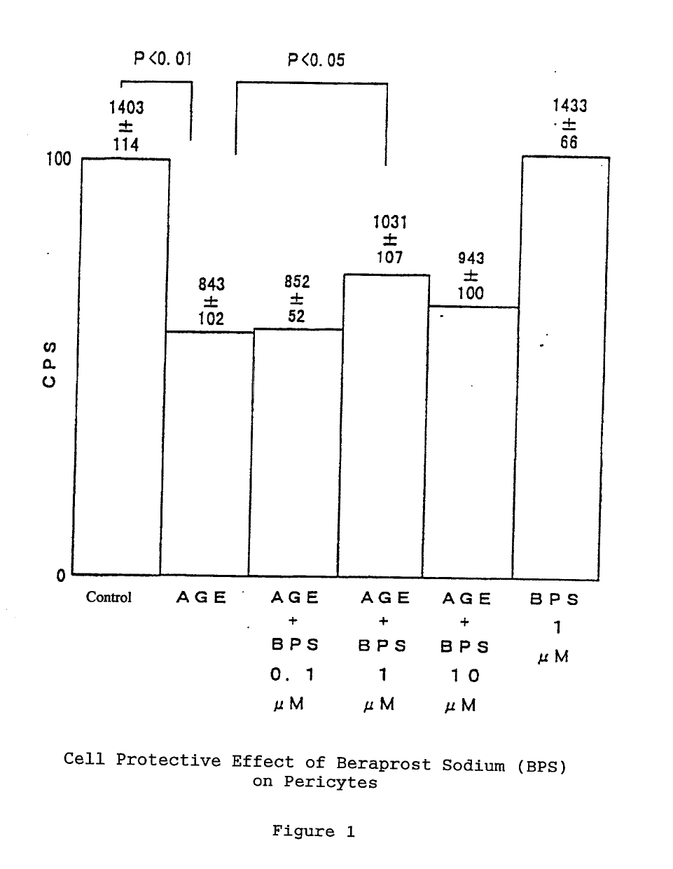

The suppression of pericyte proliferation attributable to AGE was measured by adding 0.25 mg of AGE-BSA (prepared in Reference Example 1) to a culture plate into which 3.0×10⁴ cells had been plated, then culturing at 37° C. for 5 days and performing measurements using a Packard Cyto Life. Separately, Beraprost sodium (BPS) was added at a final concentration of 0.1-10 μM prior to AGE addition. The results are shown in FIG. 1. As controls, measurements were carried out in the same way for the case when treatment was carried out with AGE-BSA only and for the case where no treatment was carried out. For each group, the tests were carried out 10 times respectively.

As shown in FIG. 1, proliferation of pericyte was significantly suppressed by AGE. As a result of BPS addition, this suppression of proliferation was improved in a concentration-dependent manner and was significant at 1 μM. Thus, based on these results, BPS exhibited a pericyte protecting action.

Next, the quantity of the AGE receptor RAGE mRNA present in the pericytes was analyzed by means of a quantitative reverse transcription-polymerase chain reaction method, in accordance with the technique described in Biochemical and Biophysical Research Communications, Vol. 213, 682-683, 1995. By simultaneously carrying out 1 μM BPS treatment, expression of RAGE was suppressed and it was concluded that suppression of the expression of RAGE, which is the AGE receptor, plays a role in the pericyte protecting action of BPS.

### Industrial Application Potential

In both oral and parenteral administration, the protective agent of the present invention has an outstanding protecting action of pericyte vascular and it is effective in the prevention and treatment of various types of vascular disorder such as diabetic retinopathy, diabetic neuropathy, diabetic nephropathy and other such diabetic angiopathy, hypertension, arteriosclerosis, peripheral circulatory disturbance (intermittent claudication and the like), cerebrovascular disorders and ischemic heart disease.

1. A pericyte protective agent having as the effective component a prostanoic acid derivative.

2. A pericyte protective agent according to claim 1 where the prostanoic acid derivative is a prostaglandin I derivative.

3. A pericyte protective agent according to claim 2 where the prostaglandin I derivative is a prostaglandin I₂ derivative.

4. A pericyte protective agent according to claim 3 where the prostaglandin I₂ derivative is a 4,8-inter-m-phenylene prostaglandin I₂ derivative represented by general formula (I) 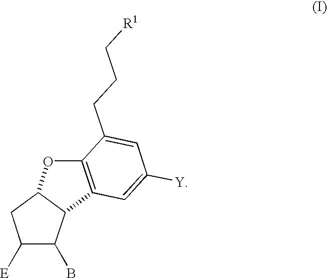
[where R¹ is
(A) COOR²
(where R² is
1) hydrogen or a pharmacologically acceptable cation,
2) a C₁₋₁₂ straight chain alkyl or C₃₋₁₄ branched alkyl,
3) -Z-R³
(where Z is a valence bond or a straight chain or branched alkylene represented by CₜH₂ₜ, where t denotes an integer in the range 1-6, and R³ is a C₃₋₁₂ cycloalkyl or C₃₋₁₂ substituted cycloalkyl which is substituted with from one to three R⁴ groups, and R⁴ denotes hydrogen or a C₁₋₅ alkyl),
4) —(CH₂CH₂O)ₙCH₃
(where n denotes an integer in the range 1 to 5),
5) -Z-Ar¹
(where Z is defined as above and Ar¹ denotes phenyl, α-naphthyl, β-naphthyl, 2-pyridyl, 3-pyridyl, 4-pyridyl, α-furyl, β-furyl, α-thienyl, β-thienyl or substituted phenyl (where the substituent groups are at least one of chlorine, bromine, fluorine, iodine, trifluoromethyl, C₁₋₄ alkyl, nitro, cyano, methoxy, phenyl, phenoxy, p-acetamidobenzamido, —CH═N—NH—C(═O)—NH₂, —NH—C(═O)-Ph, —NH—C(═O)—CH₃ or —NH—C(═O)—NH₂)),
6) —CₜH₂ₜCOOR⁴
(where CₜH₂ₜ and R⁴ are as defined above),
7) —CₜH₂ₜN(R⁴)₂
(where CₜH₂ₜ and R⁴ are as defined above),
8) —CH(R⁵)—C(═O)—R⁶
(where R⁵ denotes hydrogen or benzoyl, and R⁶ denotes phenyl, p-bromophenyl, p-chlorophenyl, p-biphenyl, p-nitrophenyl, p-benzamidophenyl or 2-naphthyl),
9) —CₚH₂ₚ—W—R⁷
(where W is —CH═CH—, —CH═CR⁷— or —C≡C—, and R⁷ is hydrogen or a C₁₋₃₀ straight chain or branched alkyl or aralkyl), and p denotes an integer in the range 1-5), or
10) —CH(CH₂OR⁸)₂
(where R⁸ denotes a C₁₋₃₀ alkyl or acyl),
(B) —CH₂OH
(C) —C(═O)N(R⁹)₂
(where R⁹ represents hydrogen, a C₁₋₁₂ straight chain alkyl, C₃₋₁₂ branched alkyl, C₃₋₁₂ cycloalkyl, C₄₋₁₃ cycloalkylalkylene, phenyl, substituted phenyl (where the substituent groups are as defined in (A) 5) above), C₇₋₁₂ aralkyl or SO₂R¹⁰, and R¹⁰ represents a C₁₋₁₀ alkyl, C₃₋₁₂ cycloalkyl, phenyl, substituted phenyl (where the substituent groups are as defined in (A) 5) above) or a C₇₋₁₂ aralkyl, and the two R⁹ groups may be the same or different, but where one represents —SO₂R¹⁰ then the other R⁹ is not —SO₂R¹⁰), or
(D) —CH₂OTHP (where THP is a tetrahydropyranyl group),
Y is hydrogen, a C₁₋₄ alkyl, chlorine, bromine, fluorine, formyl, methoxy or nitro,
B is —X—C(R¹¹)(R¹²)OR¹³
(where R¹¹ is hydrogen or a C₁₋₄ alkyl, R¹³ is hydrogen, C₁₋₁₄ acyl, C₆₋₁₅ aroyl, tetrahydropyranyl, tetrahydrofuranyl, 1-ethoxyethyl or t-butyl,
X is
1) —CH₂ —CH₂—
2) —CH═CH— or
3) —C≡C— and
R¹² represents
1) a C₁₋₁₂ straight chain alkyl or C₃₋₁₄ branched alkyl,
2) -Z-Ar²
(where Z is as defined above and Ar² represents phenyl, α-naphthyl, β-naphthyl or phenyl substituted with at least one of chlorine, bromine, fluorine, iodine, trifluoromethyl, C₁₋₄ alkyl, nitro, cyano, methoxy, phenyl or phenoxy),
3) —CₜH₂ₜOR¹⁴
(where CₜH₂ₜ is as defined above and R¹⁴ represents a C₁₋₆ straight chain alkyl, C₃₋₆ branched alkyl, phenyl, phenyl substituted with at least one of chlorine, bromine, fluorine, iodine, trifluoromethyl, C₁₋₄ alkyl, nitro, cyano, methoxy, phenyl or phenoxy, cyclopentyl, cyclohexyl, or cyclopentyl or cyclohexyl substituted with from one to four C₁₋₄ straight chain alkyls),
4) -Z-R³
(where Z and R³ are as defined above),
5) —CₜH₂ₜ—CH═C(R¹⁵)R¹⁶
(where CₜH₂ₜ is as defined above, and R¹⁵ and R¹⁶ each independently represent hydrogen, methyl, ethyl, propyl or butyl), or
6) —CᵤH₂ᵤ—C≡C—R¹⁷
(where u is an integer in the range 1-7, CᵤH₂ᵤ represents a straight chain or branched alkylene, and R¹⁷ represents a C₁₋₆ straight chain alkyl,
E represents hydrogen or —OR¹⁸ (where R¹⁸ represents a C₁₋₁₂ acyl, C₇₋₁₅ aroyl or R² (where R² is as defined above)),
and where the general formula represents the d-form, l-form or dl-form],
or pharmacologically acceptable salt thereof.

5. A pericyte protective agent according to claim 4 where the prostaglandin I derivative is Beraprost or salt thereof.

6. A pericyte protective agent according to any of claims 1 to 5 which is for protecting pericytes from pericyte loss due to advanced glycation endproducts.
